(19) United States
(12) Patent Application Publication
(10) Pub. No.: US 2026/0137402 A1
(43) Pub. Date: May 21, 2026

(54) SYSTEMS, DEVICES, AND METHODS FOR TREATING VESSEL OCCLUSIONS

(71) Applicant: Covidien LP, Mansfield, MA (US)

(72) Inventors: Danyong Zeng, Aliso Viejo, CA (US); Tejashri Kumar, Fountain Valley, CA (US); Mark P. Ashby, Laguna Niguel, CA (US); Tasha C. Aboufadel, Oceanside, CA (US); Maria D. Sanson, Mission Viejo, CA (US); Omarinda S. Nichols, Lakewood, CA (US)

(21) Appl. No.: 19/391,910

(22) Filed: Nov. 17, 2025

Related U.S. Application Data
(60) Provisional application No. 63/722,507, filed on Nov. 19, 2024.

(51) Int. Cl. A61B 17/22; A61B 17/00; A61B 90/00

(52) U.S. Cl. CPC A61B 17/22; A61B 90/39; A61B 2017/00022; A61B 2017/00199; A61B 2017/00477; A61B 2017/00561; A61B 2017/00778; A61B 2017/22038; A61B 2017/22079; A61B 2090/3966; A61B 2217/005

(57) ABSTRACT

Treatment devices and associated methods and systems are disclosed herein. According to some embodiments, the present technology includes a treatment device including a tubular member having a proximal end region configured to be disposed extracorporeally and a distal end region configured to be disposed at an intravascular treatment site. The tubular member can include a sidewall defining a lumen extending from the proximal end region to the distal end region, and a plurality of side openings in the sidewall in the distal end region.

### CROSS-REFERENCE TO RELATED APPLICATION(S)

The present application claims priority to U.S. Provisional Ser. No. 63/722,507, filed Nov. 19, 2024, which is incorporated by reference herein in its entirety.

### TECHNICAL FIELD

The present technology relates to systems and methods for removing obstructions from body lumens. Some embodiments of the present technology relate to aspiration catheters and associated components.

### BACKGROUND

Many medical procedures use medical device(s) to remove an obstruction (such as clotting material) from a body lumen, vessel, or other organ. An inherent risk in such procedures is that mobilizing or otherwise disturbing the obstruction can potentially create further harm if the obstruction or a fragment thereof dislodges from the retrieval device. If all or a portion of the obstruction breaks free from the device and flows downstream, it is highly likely that the free material will become trapped in smaller and more tortuous anatomy. In many cases, the physician will no longer be able to use the same retrieval device to again remove the obstruction because the device may be too large and/or immobile to move the device to the site of the new obstruction.

Procedures for treating ischemic stroke by restoring flow within the cerebral vasculature are subject to the above concerns. The brain relies on its arteries and veins to supply oxygenated blood from the heart and lungs and to remove carbon dioxide and cellular waste from brain tissue. Blockages that interfere with this blood supply eventually cause the brain tissue to stop functioning. If the disruption in blood occurs for a sufficient amount of time, the continued lack of nutrients and oxygen causes irreversible cell death. Accordingly, it is desirable to provide immediate medical treatment of an ischemic stroke.

To access the cerebral vasculature, a physician typically advances a catheter from a remote part of the body (typically a leg) through the abdominal vasculature and into the cerebral region of the vasculature. Once within the cerebral vasculature, the physician deploys a device for retrieval of the obstruction causing the blockage, for example an aspiration catheter. Concerns about dislodged obstructions or the migration of dislodged fragments increases the duration of the procedure at a time when restoration of blood flow is paramount. Furthermore, a physician might be unaware of one or more fragments that dislodge from the initial obstruction and cause blockage of smaller more distal vessels. Accordingly, there remains a need for improved devices and methods that can remove occlusions from body lumens and/or vessels.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a side view of an example treatment system in accordance with examples of the present technology.

FIGS. 2A and 2B are graphs of pressure drops over various catheter lengths as a function of inner diameter in accordance with embodiments of the present technology.

FIG. 3 is a graph of stiffness over length for a catheter in accordance with embodiments of the present technology.

FIG. 4A is a side view of an example distal shaft for a treatment device in accordance with embodiments of the present technology.

FIG. 4B is a side cross-sectional view of the distal shaft shown in FIG. 4A.

FIG. 4C is an enlarged cross-sectional side view of the distal tip of the distal shaft shown in FIGS. 4A and 4C.

FIG. 5A is a side view of an example distal shaft for a treatment device in accordance with embodiments of the present technology.

FIG. 5B is a side cross-sectional view of the distal shaft shown in FIG. 5A.

FIG. 6A is a perspective view of an example distal shaft for a treatment device in accordance with embodiments of the present technology.

FIG. 6B is a side cross-sectional view of the distal shaft of the treatment device shown in FIG. 6A, taken along a first axis.

FIG. 6C is side cross-sectional view of the distal shaft shown in FIG. 6A, taken along a second axis orthogonal to the first.

FIGS. 7A-7F are side views of example treatment devices with various side opening arrangements in accordance with embodiments of the present technology.

FIGS. 8A-8E are side views of example sidewall opening configurations in accordance with embodiments of the present technology.

FIG. 9A is a side view of a distal tip region of an example treatment device with a guidewire extending therethrough in accordance with embodiments of the present technology.

FIG. 9B is a side cross-sectional view of the distal tip region shown in FIG. 9A.

FIG. 10 is a side view of a portion of a treatment device with an example joint connecting the proximal shaft and the distal shaft in accordance with examples of the present technology.

FIG. 11A shows a partially exploded view of another example joint of a treatment device in accordance with examples of the present technology.

FIG. 11B shows a side view of the joint shown in FIG. 11A in an assembled state.

FIG. 12 shows a perspective view of another example joint of a treatment device in accordance with examples of the present technology.

FIG. 13 shows a side view of another example joint of a treatment device in accordance with examples of the present technology.

FIGS. 14-16 are side views of treatment devices having radiopaque markers in various configurations in accordance with examples of the present technology.

FIG. 17 is a side perspective view of an example radiopaque marker in accordance with examples of the present technology.

FIG. 18 is a side perspective view of another example radiopaque marker in accordance with examples of the present technology.

FIGS. 19A-19D illustrate an example method of thrombectomy using a treatment device in accordance with examples of the present technology.

FIG. 20 is a graph of measured flow rate during use of a treatment system under various clot-engagement conditions in accordance with examples of the present technology.

FIGS. 21 and 22 show example images of the treatment device engaged with a clot in a test configuration.

### DETAILED DESCRIPTION

The present technology relates to systems and methods for removing obstructions from body lumens. Some embodiments of the present technology relate to aspiration catheters and associated components. In various examples, a treatment device takes the form a catheter having a proximal end configured to be positioned extracorporeally and a distal region configured to be positioned at an intravascular treatment site. The catheter may further include a sidewall defining a lumen extending along the length of the catheter. A plurality of sidewall openings can be formed in the catheter along the distal region which are in fluid communication with the lumen. In operation, when a suction is applied to the lumen (e.g., via an extracorporeal suction source coupled fluidically coupled to the lumen), these sidewall openings serve as aspiration openings and can pull fluid therethrough and into the lumen and/or pull clot material into engagement with the treatment device. Once the clot is engaged with the treatment device (and secured in place via the negative pressure applied through the sidewall openings), the treatment device and clot can be removed from the vessel (e.g., slidably retracted), thereby removing the occlusion and restoring blood flow. Additional details regarding the use of catheters with sidewall openings (also referred to as a “perforated catheter”) can be found in U.S. patent application Ser. No. 18/435,846, titled “Catheter Defining Longitudinally Arranged and Fluidically Connected Openings at a Distal Region,” filed Feb. 7, 2024, which is hereby incorporated by reference in its entirety for all purposes.

The systems, devices, and methods of the present technology can provide many advantages compared to conventional devices and techniques for treating vascular obstructions, particularly in the context of smaller vessels such as those of the cerebral vasculature. For larger vessels, a relatively simple large-bore catheter with a single distal opening may be suitable for aspiration to remove occlusions. However, to access treatment sites in smaller vessels, microcatheters having outer smaller outer diameters (e.g., about 1.7 French to about 2.7 French) are required, and these microcatheters have correspondingly smaller inner diameters (e.g., about 0.010 inches to about 0.027 inches). As a result of these smaller lumen diameters, there is a high degree of pressure drop from the proximal end of the catheter to the distally located treatment site. This problem is exacerbated by the need for relatively long lumens in neurovascular applications (e.g., about 1.3 m to about 1.6 m in length). The resulting pressure drop makes it difficult to effectively deliver sufficient negative pressure to the treatment site to aspirate the clot.

Another problem with conventional aspiration catheters in the context of small-vessel occlusions is that the small lumens required for such procedures corresponds to a small opening at the distal face of the catheter, and this renders it difficult to effectively engage the clot. This can result in “corking,” in which clot material completely or substantially blocks the distal opening of the catheter but is not drawn further into the lumen. Additionally, this single point of engagement between the clot and the aspiration catheter renders the clot more likely to dislodge from the catheter, or to fragment so that a portion of the clot remains engaged but other portions break way and drift downstream, posing the risk of distal embolism.

The treatment systems and methods described herein address these and other problems of conventional aspiration catheters. Among other features, various embodiments of the present technology include catheters having a plurality of sidewall openings that can be disposed radially adjacent to clot material within a blood vessel. Upon supplying suction to the lumen, the clot is pulled into engagement with one or more of the sidewall openings that face or are otherwise aligned with the clot, which can provide multiple points of engagement and enhance the grip on the clot. Additionally or alternatively, the treatment device can have a multi-staged diameter such that the outer dimension and/or lumen tapers distally, with gradual transition zones between stages (or alternatively a relatively continuous taper along some or all of the length). This can facilitate pushability (increased column strength due to larger outer diameter), avoidance of the “ledge effect” when navigating tortuous anatomy (due to larger outer diameter and gradual transition zones), and increased delivery of suction force (due to less pressure drop across the length of the catheter as a result of the larger lumen size along at least a proximal portion of the catheter).

As one example, an aspiration catheter can have a proximalmost segment having a relatively large lumen dimension (e.g., about 0.071 inches), which may be stepped down in stages via gradual transition zones, for instance first to an intermediate segment having a smaller lumen dimension (e.g., about 0.055 inches). And at a distal region of the treatment device, the lumen can taper still further down along the treatment site where the sidewall openings are located (e.g., to about 0.027″). Optionally, the lumen can taper still further to a distal tip (e.g., to about 0.010 inches) to a size just large enough to slidably accommodate a guidewire therethrough.

Various other features and aspects of example treatment systems and devices are described in more detail below. As described in more detail below, particular features of the treatment device can further optimize operation of the device, engagement with the clot, navigability to small or tortuous vessels, or other aspects. Such features include the number, size, shape, and arrangement of the sidewall openings, the shape, size, and configuration of the various regions along the catheter, the mechanisms for joining shafts together to form the assembled catheter, the shape, arrangement, and configuration of radiopaque markers along the treatment device. The treatment systems and devices described herein also enable beneficial methods as described in more detail below.

### I. Example Treatment Systems and Devices

a. Treatment System Overview

The present technology provides systems, devices, and methods for treating various medical conditions, including removing a thrombus or other occlusion from a bodily lumen. Although many of the embodiments are described below with respect to devices, systems, and methods for treating a cerebral or intracranial embolism, other applications and other embodiments in addition to those described herein are within the scope of the technology. For example, the treatment systems and methods of the present technology may be used to remove emboli from body lumens other than blood vessels (e.g., the digestive tract, etc.) and/or may be used to remove emboli from blood vessels outside of the brain (e.g., pulmonary, abdominal, cervical, or thoracic blood vessels, or peripheral blood vessels including those within the legs or arms, etc.). In addition, the thrombectomy systems and methods of the present technology may be used to remove luminal obstructions other than clotting material (e.g., plaque, resected tissue, foreign material, etc.). Additionally or alternatively, the treatment systems and devices described herein can be used for fluid delivery to a treatment site within a bodily lumen (e.g., delivery of medicament, saline, contrast media, or other suitable fluid to an intravascular treatment site within a cerebral vessel or other desired treatment site).

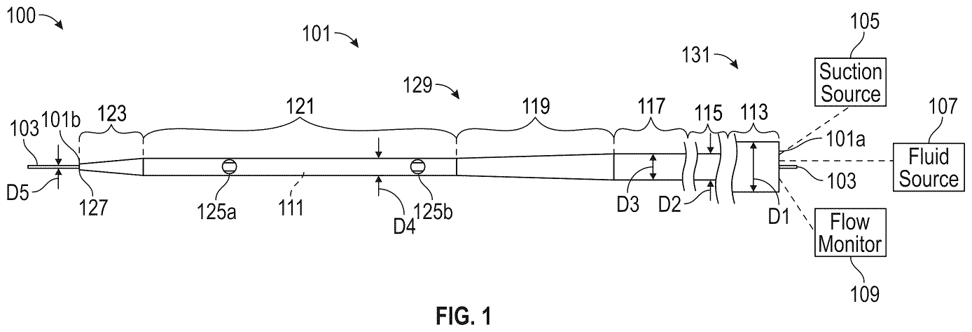

FIG. 1 illustrates a perspective view of a treatment system 100, in accordance with embodiments of the present technology. As shown in FIG. 1, the treatment system 100 can include a treatment device 101 (e.g., a tubular member, a catheter, etc.), a guidewire 103 configured to slidably extend along the length of the treatment device 101, and, optionally, various extracorporeal components such as suction source 105, fluid source 107, and flow monitor 109 that are configured to be fluidically coupled to the treatment device 101. The treatment device 101 includes an interior lumen 111 that extends from the proximal end 101a to the distal end 101b of the treatment device 101. In operation, the suction source 105, the fluid source 107, and/or the flow monitor 109 can be fluidically coupled to the lumen 111 at or near the proximal end 101a of the treatment device 101, for instance via a hub, port, or handle (not shown).

The guidewire 103 can be sized and configured to be slidably disposed within the lumen 111 of the treatment device 101. In various embodiments, the guidewire 103 can be a solid pushwire or guidewire. Additionally or alternatively, the guidewire 103 can instead include a hollow wire, hypotube, braid, coil, or other suitable member(s), or a combination of wire(s), tube(s), braid(s), coil(s), etc. In some embodiments, the guidewire 103 can be made of stainless steel (e.g., 304 SS), Nitinol, and/or other alloy. In operation, to deploy the treatment device 101 at an intravascular treatment site (e.g., at or adjacent to a thrombus), the guidewire 103 may first be advanced at or near the treatment site, after which the treatment device 101 can be slidably advanced over the guidewire 103, which is received within the lumen 111 of the treatment device 101. The guidewire 103 can then be removed after the treatment device 101 is positioned at the intravascular treatment site.

The suction source 105 can be pump, syringe, etc. configured to supply negative pressure to the lumen for aspiration, as described in more detail below. The fluid source 107 can be pump, syringe, etc. coupled to or including a reservoir containing a fluid (e.g., saline, medicament, contrast media, etc.) configured to be delivered through the lumen for delivery to a treatment site. This involves delivery of positive pressure through the lumen 111, in contrast to the suction applied via the suction source 105.

The flow monitor 109 includes a sensor configured to obtain measurements indicative of fluid flow through the lumen 111. These can indicate efficacy of aspiration, fluid delivery, or other aspects of device operation. According to some implementations, the flow monitor 109 can incorporate various types of flow sensing technologies. For instance, the flow monitor 109 can include ultrasonic flow sensors that measure flow rate using transit-time or Doppler-based measurements. Additionally or alternatively, the flow monitor 109 can incorporate thermal mass flow sensors that detect flow rates based on heat transfer characteristics of the flowing fluid. In some examples, electromagnetic flow meters that measure voltage induced by conductive fluid passing through a magnetic field can be used. The flow monitor 109 can also include differential pressure sensors that determine flow rate based on pressure differences across a known restriction, or rotameters that measure flow using a float suspended in a tapered tube.

In various implementations, the flow monitor 109 can be configured to provide real-time feedback about device operation. For instance, during aspiration procedures, measured flow rates can indicate the degree of engagement between the treatment device and surrounding tissue. According to some examples, flow rates approaching zero can indicate substantial engagement between a clot and the sidewall openings, while increased flow rates can suggest partial or complete disengagement. Additionally or alternatively, the flow monitor 109 can help confirm proper fluid delivery during infusion procedures, with measured flow rates indicating whether target delivery volumes are being achieved. The flow monitor 109 can include processing components configured to analyze the flow measurements and provide feedback to the physician, such as visual displays, audible alerts, or other suitable indicators of device operation status.

In some implementations, the flow monitor 109 can be calibrated to account for different fluid properties, temperature variations, or other factors that might affect flow measurements. The flow monitor 109 can optionally include data logging capabilities to record flow measurements over time, which may be useful for procedure documentation or analysis. According to various examples, the flow monitor 109 can be configured to detect sudden changes in flow rate that might indicate complications such as device occlusion, vessel perforation, or other issues requiring immediate attention.

The treatment device 101 can include a plurality of different regions with different material and/or dimensional properties. In general, both the radially outermost dimension of the treatment device 101 and the dimension of the lumen 111 vary along the length of the treatment device 101, and can generally taper distally, such that the greater dimensions are arranged near the proximal end 101a and get progressively smaller toward the distal end 101b. In the illustrated example, the treatment device 101 includes the following regions (from proximal to distal): proximal region 113, intermediate region 115, interface region 117, transition region 119, treatment region 121, and distal tip region 123. As illustrated, the outer dimensions of the lumen 111 can vary along the length in these various regions, from dimension D1 in the proximal region 113, to dimension D2 in the intermediate region 115, dimension D3 in the interface region 117, dimension D4 in the treatment region 121, and terminating in dimension D5 at the distal end of the distal tip region 123. In some embodiments, each subsequent dimension, from proximal to distal, is increasingly smaller. This gradual tapering of the lumen dimension allows for a larger to be accommodated where feasible given access constraints (e.g., in the proximal region 113 and intermediate region 115) while tapering down to smaller dimensions where needed to facilitate access to distal locations, such as those within the cerebral vasculature. Although six discrete regions are depicted in this example, in various implementations there may be more or fewer. For instance, the distal tip regio 123 can be omitted in some instances (i.e., the material and dimensional properties of the treatment region 121 can continue to the distal terminus). Additionally or alternatively, the intermediate region 115 can be omitted, in which case the proximal region 113 can interface with (and transition smoothly to) the interface region 117. In some implementations, instead of a single intermediate region 115 between the proximal region 113 and the interface region 117, there may be 2, 3, 4, 5, 6 or more different intermediate regions, each having progressively smaller outer and/or luminal dimensions.

As shown in FIG. 1, within the treatment region 121 a plurality of side openings 125a and 125b (collectively “sidewall openings 125”) are formed in the sidewall of the treatment device 101. These sidewall openings 125 place the lumen 111 in fluid communication with the environment surrounding the treatment device 101 along the treatment region 121 (e.g., a blood vessel). The side openings 125 can include any variety of sizes, arrangements, or geometries. For instance, the side openings 125 can be or include circular, straight, arcuate, curved, semi-circular, or semi-elliptical shapes. The side openings 125 may optionally be or include complex shapes, such as zig-zag, undulating, serpentine, sinusoidal, or a combination thereof. In some implementations, the side openings 125 can take the form of windows, apertures, voids, cuts, or other such structures that allow fluid to pass therethrough. The side openings 125 can be arranged with longitudinal spacing and/or radial spacing. In the example shown in FIG. 1, the two side openings 125a and 125b are aligned along the longitudinal axis such that they are both disposed on the same radial face of the treatment device 101. In various implementations, however, different side openings 125 may be spaced apart radially, such as being arranged on opposing radial side of the treatment region 121. Further, side openings 125 may be arranged in other configurations, such as in a grid, array, in a spiral around the treatment device 101, or any other suitable arrangement. The treatment device 101 can further include an opening 127 at the end of the distal tip region 123 through which the guidewire 103 can pass. In various examples, a distal end of the side openings 125 and the distal opening 127 can be separated from one another along the longitudinal axis of the treatment device 101 by at least 1 mm, 5 mm, 10 mm, 20 mm, 50 mm, 100 mm, 1 cm, 5 cm, 10 cm, etc. The number of openings 125 can vary, for example, 1, 2, 3, 4, 5, 6, 7, 8, 9, 10 or more openings 125 can be provided along the treatment region 121.

In various implementations, the treatment device 101 can take the form of a tubular member such as a catheter (e.g., an aspiration catheter), and can be made of a single unitary body or composed of a plurality of separate components coupled together to define the resulting treatment device 101. For instance, in some instances the treatment device 101 can take the form of a distal shaft 129 coupled to one or more proximal shafts 131. In the illustrated example, the distal shaft 129 can be made of a more flexible material (e.g., silicone or other suitable elastomeric material), and can include the interface region 117, the transition region 119, the treatment region 121, and the distal tip region 123. The proximal shaft(s) 131 can take the form of more conventional catheter constructions, such as tubular members reinforced with metallic hypotubes or metallic braids. To assemble the two, the interface region 117 of the distal shaft 129 is configured to interface and/or interlock with the distal end of the proximal shaft 131, as described in more detail below. The relatively stiffer and larger diameters of the proximal shaft(s) 131 can be fluidically coupled to the distal shaft 129 to produce the treatment device 101. In some examples, there are two proximal shafts 131: a first corresponding to the proximal region 113 (e.g., a tubular member having an inner diameter of D1) and a second corresponding to the intermediate region 115 (e.g., a tubular member having an inner diameter of D2). These two proximal shafts can be joined together via a transition (not illustrated), for instance having a gradual taper or other such transition between different outer and inner dimensions of these tubular members. In alternative configurations, there may be a single proximal shaft 131, which can have a substantially constant inner diameter or having an inner diameter that tapers distally along or some or all of its length.

In operation, the treatment device 101 can be used as an aspiration catheter to remove a clot or other material such as plaques or foreign bodies from vasculature of a patient. For example, a vacuum may be applied to a proximal end 101a of the treatment device 101 (e.g., via suction source 105) to draw a clot or other blockage into an inner lumen 111 of the treatment device 101. In some embodiments, the vacuum causes the clot or other blockage to remain attached to the treatment device 101 (e.g., on an outer surface of the treatment device 101). The treatment device 101 with the engaged clot adhered thereto may be slidably retracted from the treatment site and removed from the body. Optionally, the treatment device 101 with the engaged clot adhered thereto can be pulled into a surrounding catheter (e.g., a guide catheter, access catheter, etc.) or may be retracted completely from the body without the use of a separate surrounding catheter. Such aspiration may be used in various medical procedures, such as a medical procedure to treat an ischemic insult, which may occur due to occlusion of a blood vessel (arterial or venous) that deprives brain tissue, heart tissue or other tissues of oxygen-carrying blood.

With continued reference to FIG. 1, in some examples, the treatment device 101 can be configured to access relatively distal locations in a patient including, for example, the middle cerebral artery (MCA), internal carotid artery (ICA), the Circle of Willis, and tissue sites more distal than the MCA, ICA, and the Circle of Willis. The MCA, as well as other vasculature in the brain or other relatively distal tissue sites (e.g., relative to the vascular access point), may be relatively difficult to reach with a catheter, due at least in part to the tortuous pathway (e.g., comprising relatively sharp twists or turns) through the vasculature to reach these tissue sites. As such, the tubular body of the treatment device 101 may be structurally configured to be relatively flexible, pushable, and relatively kink-and buckle-resistant, so that it may resist buckling when a pushing force is applied to a relatively proximal section of the tubular member to advance the treatment device 101 distally through vasculature, and so that it may resist kinking when traversing around a tight turn in the vasculature. In some examples, the treatment device 101 is configured to substantially conform to the curvature of the vasculature. In addition, in some examples, the tubular member has a column strength and flexibility that allow at least a distal portion of the tubular member to be navigated from a femoral artery, through the aorta of the patient, and into the intracranial vascular system of the patient, e.g., to reach a relatively distal treatment site. In the case of neurovascular procedures, the treatment device 101 can have a length that is at least 125 cm long, and more particularly may be between about 125 cm and about 175 cm long.

Although primarily described as being used to reach relatively distal vasculature sites, the treatment device 101 may also be configured to be used with other target tissue sites. For example, treatment device 101 may be used to access tissue sites throughout the coronary and peripheral vasculature, the gastrointestinal tract, the urethra, ureters, fallopian tubes, veins and other body lumens.

According to some embodiments, at least some of the treatment device 101 can be made from various polymers, such as silicone or other suitable elastomeric materials, as well as various thermoplastics, e.g., polytetrafluoroethylene (PTFE or TEFLON®), fluorinated ethylene propylene (FEP), high-density polyethylene (HDPE), polyether ether ketone (PEEK), etc., which can optionally be lined on the inner surface of the catheters and/or tubular member or an adjacent surface with a hydrophilic material such as polyvinylpyrrolidone (PVP) or some other plastic coating. Additionally, surfaces can be coated with various combinations of different materials, depending upon the desired results.

As described in more detail below, some or all of the proximal region 113 and/or the intermediate region 115 can be formed of a metallic material, such as Nitinol, stainless steel, or other suitable material. In some examples, at least a portion of the treatment device 101 can include a laser-cut hypotube having a pattern of cut voids (e.g. spiral cut, separated slot cuts, or other suitable pattern) formed in its sidewall along at least a portion of its length, a metallic-braid-reinforced tubular member, or other such structure. In at least some embodiments, the treatment device 101 can have a laser cut pattern to achieve the desired mechanical characteristics (e.g., column strength, flexibility, kink-resistance, etc.).

### A. Telescopic Configuration for Improved Suction Force and Navigability

In various implementations, the treatment device 101 includes a multi-staged configuration in which both the outer dimension and lumen dimension vary along the length of the device. For example, a treatment device 101 can include a proximal region 113 having a first lumen dimension D1, an intermediate region 115 having a second lumen D2 dimension smaller than the first lumen dimension, and one or more distal regions region having a third lumen dimension (e.g., treatment region 121 having lumen dimension D4) smaller than the second lumen dimension D2. The transitions between these regions can be gradual to facilitate navigation through tortuous anatomy.

This staged configuration can provide several advantages compared to conventional devices having a constant lumen dimension. For instance, in neurovascular applications, treatment devices often need to navigate from a remote access point through progressively smaller vessels to reach a treatment site in the cerebral vasculature. The multi-staged configuration allows the distal region to have a reduced profile suitable for accessing small-diameter vessels while the proximal region can maintain a larger lumen dimension where anatomically feasible.

In some implementations, the larger lumen dimension in the proximal region reduces the pressure drop that occurs when applying suction through the device. According to some examples, negative pressure applied at the proximal end of the treatment device experiences less reduction over the length of the larger-dimensioned proximal region compared to conventional devices having a constant small diameter. As a result, more of the supplied suction force can be delivered to the treatment site. Additionally or alternatively, the reduced profile of the distal region can help the device navigate to and through narrow or tortuous vessels that may be inaccessible to larger devices.

According to some implementations, the treatment device 101 includes gradual transitions between regions having different dimensions. For example, the intermediate region can include a taper between the first and second lumen dimensions D1 and D2, and so forth. These gradual transitions can help avoid abrupt steps or “ledges” that might impede advancement of the device through curved vessels. Traditional catheters have distal ends that terminate at the catheter's full ID. Such catheters may be advanced over a guidewire and or over a smaller catheter. In these cases, there is an abrupt transition in the form of a gap between the guidewire OD and/or the smaller catheter OD and the catheter ID. As the catheter is advanced over the guidewire or smaller catheter and through the vasculature, particularly when attempting to advance it into or past a vessel side branch, the abrupt transition can impede the catheter from advancing. In some examples, the outer diameter and lumen dimension may taper substantially continuously along some or all of the length of the device.

The dimensions of the various regions can be selected based on factors such as the intended use case, target anatomy, and desired flow characteristics. As one example, the proximal region 113 can have a lumen dimension between about 0.044 inches and about 0.088 inches, the intermediate region 115 can taper to a lumen dimension between about 0.035 inches and about 0.075 inches, and the distal regions (e.g., interface region 117, transition region 119, treatment region 121, and distal tip region 123) can further taper to a lumen dimension between about 0.010 inches and about 0.055 inches. These dimensions are provided as examples only, and other suitable dimensions may be selected for particular applications.

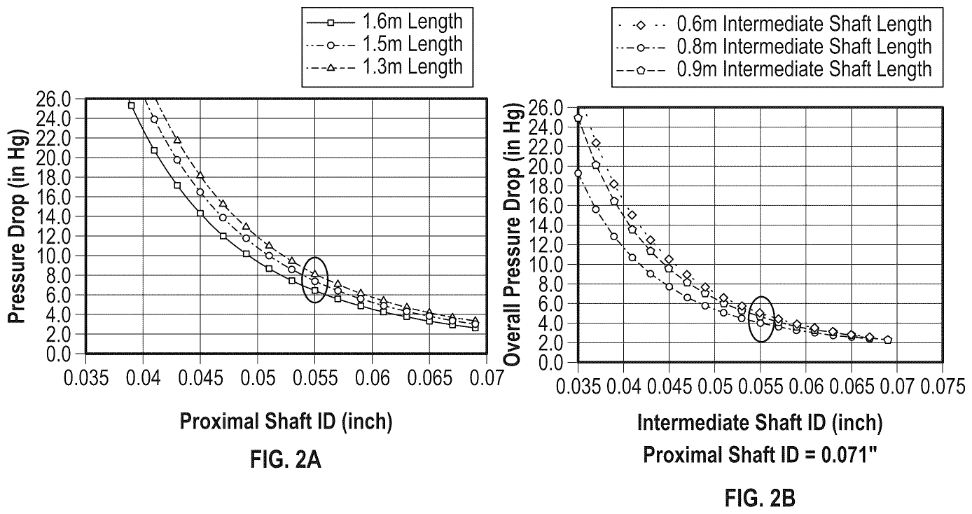

FIGS. 2A and 2B are graphs of pressure drops over various catheter lengths as a function of inner diameter. FIG. 2A shows pressure drop as a function of proximal shaft inner diameter for three different total catheter lengths (1.3 m, 1.5 m, and 1.6 m). FIG. 2B shows pressure drop as a function of intermediate shaft inner diameter, where the proximal shaft inner diameter is fixed at 0.071 inches, for three different intermediate shaft lengths (0.6 m, 0.8 m, and 0.9 m).

In the illustrated example, comparing the pressure drop at an inner diameter of 0.055 inches (circled in both figures) demonstrates an advantage of the multi-stage configuration. In FIG. 2A, when using a single shaft with 0.055-inch inner diameter along the entire length, the pressure drop ranges from approximately 6-8 inHg depending on the overall catheter length. In contrast, FIG. 2B shows that when using a 0.055-inch intermediate shaft in combination with a larger 0.071-inch proximal shaft, the total pressure drop is reduced to approximately 4-5 inHg depending on the intermediate shaft length.

This reduction in pressure drop can be attributed to the larger inner diameter (0.071 inches) of the proximal shaft in the multi-stage configuration. According to some implementations, fluid flow experiences less resistance through the larger-diameter proximal portion compared to a configuration where the entire length has the smaller 0.055-inch diameter. As a result, more of the applied suction force can be transmitted to the distal end of the catheter.

Additionally or alternatively, the graphs indicate that pressure drop increases with overall catheter length in both configurations. However, the multi-stage configuration of FIG. 2B shows less sensitivity to length variations compared to the single-diameter configuration of FIG. 2A, as evidenced by the closer spacing between curves in FIG. 2B. This characteristic may provide more consistent performance across different anatomical requirements that necessitate varying catheter lengths.

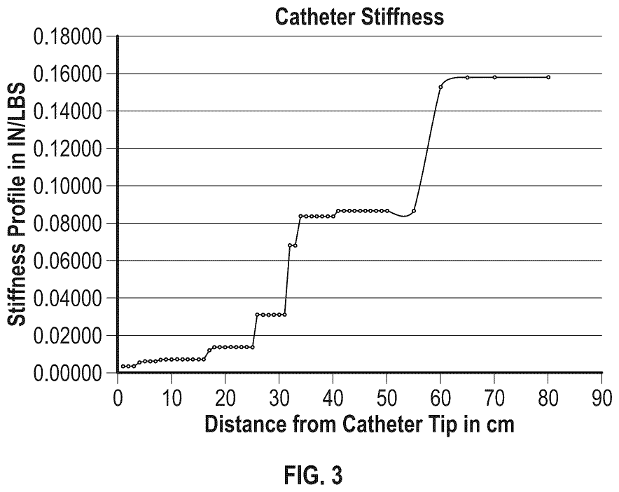

FIG. 3 is a graph of stiffness over length for an example catheter such as the treatment device 101 as measured from the distal tip. According to some implementations, the stiffness profile includes distinct regions with different stiffness characteristics, as indicated by the step-wise progression in the graph moving from distal to proximal.

In some implementations, the stiffness varies from approximately 0.005 N/LBS near the distal tip to approximately 0.155 N/LBS at the proximal end. This variation in stiffness can be achieved through multiple design features. For example, the treatment device can incorporate different wall thicknesses, varying outer dimensions, or different material compositions along its length. Additionally or alternatively, in regions incorporating metallic components, the stiffness can be modulated by varying the configuration of metallic reinforcement structures.

According to some implementations, proximal regions (e.g., proximal region 113, intermediate region 115, and/or proximal shaft 131) can include a metallic hypotube with a spiral cut pattern, where the spacing, depth, or geometry of the spiral cuts can be adjusted to achieve desired stiffness characteristics. Interrupted spirals or other patterns reducing stiffness while still capable of transmitting torque, can be used. In other examples, the proximal regions can include a metallic braid reinforcement, where the braid pitch, wire diameter, or wire density can be varied to modify local stiffness properties.

The stepped profile shown in FIG. 3 indicates discrete transitions between regions of different stiffness. These transitions can correspond to changes in structural features, such as transitions between different materials, introduction or modification of reinforcement structures, or changes in wall thickness. The gradual plateaus between transitions suggest regions of consistent construction maintaining uniform mechanical properties over those lengths.

This variable stiffness profile can provide a combination of pushability in proximal regions, where greater stiffness aids in advancing the treatment device, while maintaining flexibility in distal regions to facilitate navigation through tortuous vasculature. The progressive increase in stiffness from distal to proximal regions can help prevent kinking while allowing the distal portion to conform to vessel anatomy.

### B. Example Distal Shaft Configurations

According to various implementations, the treatment device 101 can include a distal shaft having 129 one of several configurations, each offering particular combinations of features suited for different use cases. The distal shaft 129 configurations described herein are designed to maintain a suitably low profile for navigation through small-diameter vessels while providing effective aspiration capabilities. In some implementations, the distal shaft 129 can incorporate varying degrees of taper, different cross-sectional shapes, and various wall thickness transitions to achieve desired combinations of flexibility, trackability, and aspiration performance. As described in more detail below, example configurations include combinations of tapered and straight sections, dual-tapered arrangements, and oval cross-sectional geometries, each of which may be particularly suited for specific anatomical constraints or treatment objectives. As noted previously, the distal shaft 129 can be relatively flexible, for instance being made of an elastomeric polymer such as silicone. This can be contrasted with a more rigid proximal shaft 131, which in some implementations includes stiffer structural reinforcement such as a metallic hypotube, metallic tubular braid or mesh, etc.

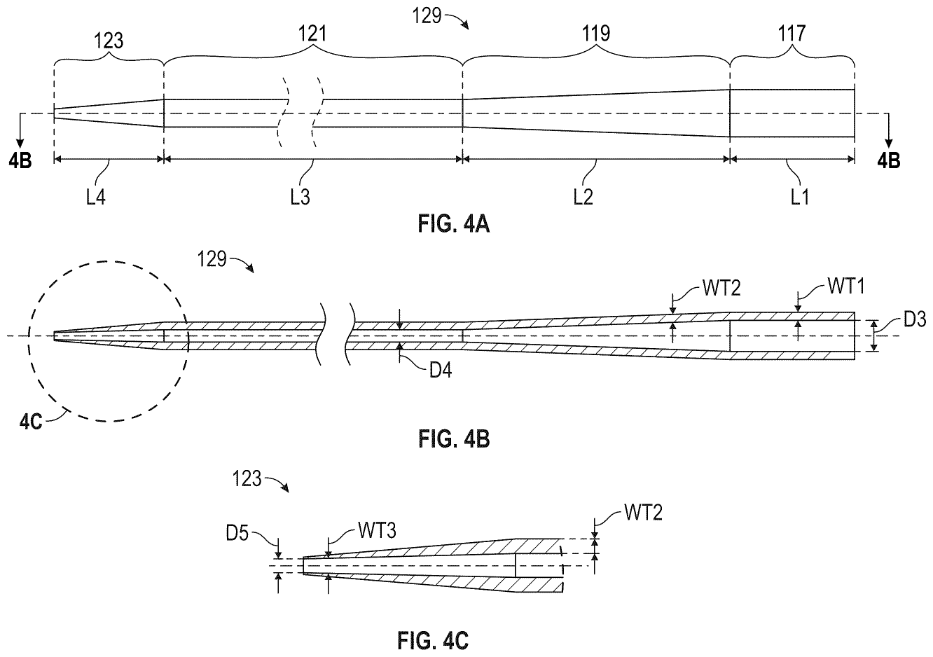

FIGS. 4A-4C illustrate various views of a distal shaft 129 according to some implementations of the present technology. FIG. 4A shows a side view of the distal shaft 129, which includes multiple regions progressing from proximal to distal: an interface region 117, a transition region 119, a treatment region 121, and a distal tip region 123. According to some implementations, these regions have lengths L1, L2, L3, and L4 respectively, where L1 is about 5 mm, L2 is about 5 mm, L3 is about 20 mm, and L4 is about 3 mm. Although the treatment region 121 here is shown without sidewall openings 125, in operation one or more such openings 125 are formed along the treatment region 121 during manufacture of the treatment device 101.

FIG. 4B provides a side cross-sectional view of the distal shaft 129, illustrating internal dimensional characteristics. In some implementations, the lumen dimension transitions from a first inner diameter D3 of about 0.052 to 0.056 inches in the interface region 117, to a second inner diameter D4 of about 0.027 inches in the treatment region 121. The wall thickness can vary along the length, transitioning from a first wall thickness WT1 of about 0.005 to 0.010 inches to a second wall thickness WT2 of about 0.004 to 0.005 inches.

FIG. 4C shows an enlarged cross-sectional view of the distal tip region 123. According to some implementations, this region includes a further reduction in lumen dimension to a third inner diameter D5 of about 0.010 inches. The wall thickness in the distal tip region can decrease to a third wall thickness WT3 of about 0.004 inches.

In some implementations, the distal shaft 129 can be formed from a material having a durometer of about 65 D to about 80 D. The progressive reduction in both lumen dimension and wall thickness from proximal to distal end can provide a combination of pushability and flexibility suited for navigating tortuous vasculature while maintaining sufficient column strength for device advancement.

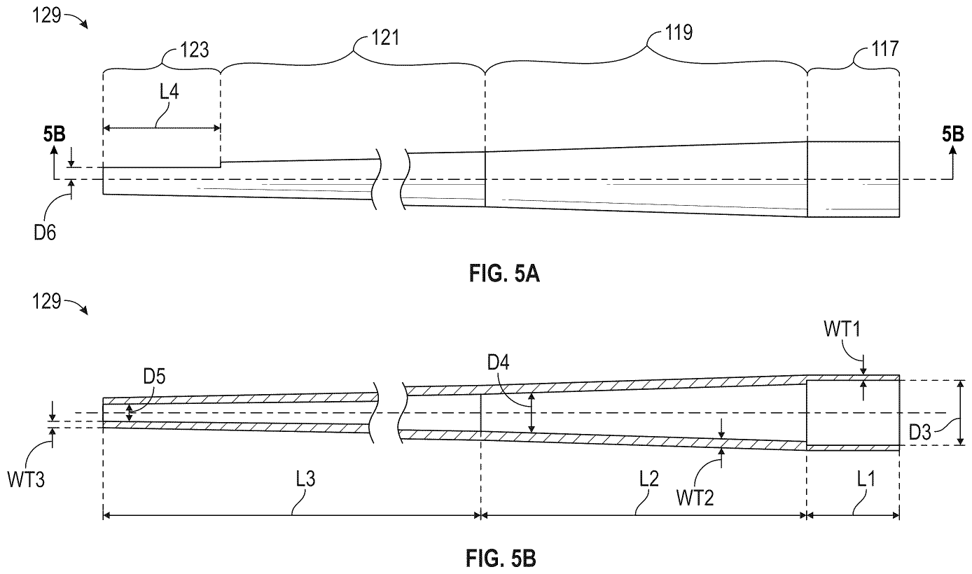

FIGS. 5A and 5B illustrate another example configuration of a distal shaft 129. FIG. 5A shows a side view of the distal shaft 129, which includes, from proximal to distal: an interface region 117, a transition region 119, a treatment region 121, and a distal tip region 123. According to some implementations, these regions have lengths L1, L2, L3-L4 (L3 is a length of both the treatment region 121 and distal tip region 123 combined), and L4, respectively, where L1 is about 5 mm, L2 is about 7-10 mm, L3 is about 14-25 mm, and L4 is about 0.1 inches. Although the treatment region 121 here is shown without sidewall openings 125, in operation one or more such openings 125 are formed along the treatment region 121 during manufacture of the treatment device 101.

FIG. 5B provides a side cross-sectional view of the distal shaft 129, illustrating the internal dimensions and wall thickness characteristics. In some implementations, the lumen transitions from a first inner diameter D3 of about 0.066 inches in the interface region 117, to a second inner diameter D4 of about 0.030 inches in the treatment region 121, and further to a third inner diameter D5 of about 0.016 inches. The distal tip can include a fourth inner diameter D6 of about 0.011 inches.

According to some implementations, the wall thickness varies along the length of the distal shaft 129. The wall thickness can transition from a first wall thickness WT1 of about 0.004 to 0.007 inches in a proximal portion to a second wall thickness WT2 of about 0.0085 to 0.010 inches in an intermediate portion. A third wall thickness WT3 of about 0.004 to 0.006 inches can be provided in a distal portion. In some implementations, the distal shaft 129 can be formed from a material having a durometer of about 65 D or about 80 D.

The progressive reduction in lumen dimension combined with the varying wall thickness can provide a balance of structural support and flexibility along the length of the distal shaft 129. These dimensional transitions can facilitate both device advancement and navigation through tortuous anatomy while maintaining desired aspiration characteristics.

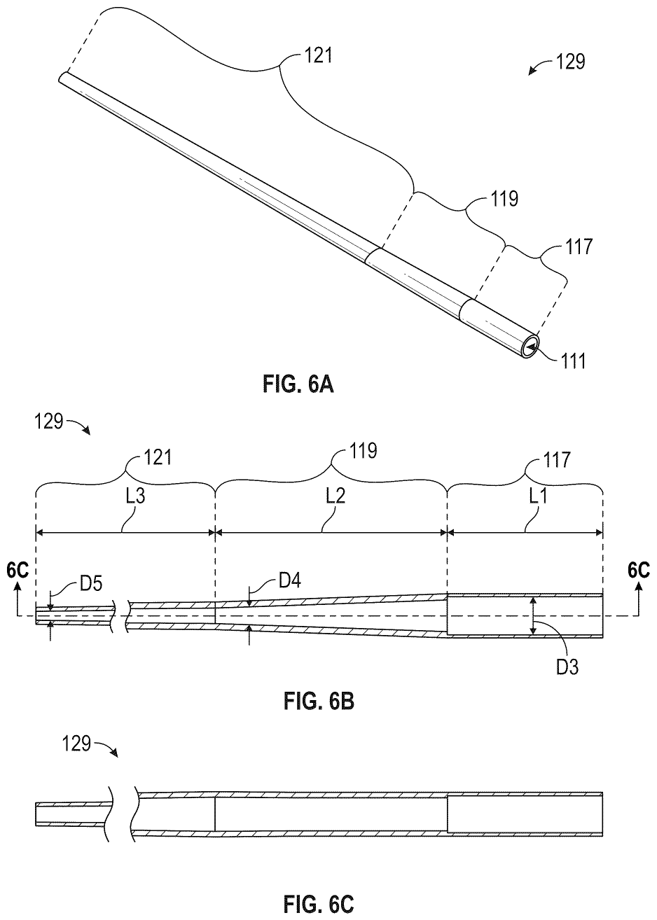

FIGS. 6A-6C illustrate views of a distal shaft 129 incorporating an oval cross-sectional geometry according to some implementations. FIG. 6A shows a perspective view of the distal shaft 129, which includes an interface region 117, a transition region 119, and a treatment region 121. According to some implementations, these regions have lengths L1, L2, and L3, respectively, where L1 is about 5 mm, L2 is about 5 -7.5 mm, and L3 is about 20-25 mm.

FIG. 6B shows a cross-sectional view taken along a first axis, while FIG. 6C shows a cross-sectional view taken along a second axis orthogonal to the first, illustrating the oval-shaped configuration. In some implementations, the lumen 111 transitions from a circular cross-section having an inner diameter D3 of about 0.037 to 0.063 inches in the interface region 117 to an oval cross-section in the transition region 119. The oval cross-section can have various dimensional configurations in dimension D4, including about 0.045 inches by 0.030 inches, about 0.040 inches by 0.025 inches, about 0.044 inches by 0.021 inches, or about 0.041 inches by 0.021 inches.

According to some implementations, the lumen 111 further transitions to a smaller oval cross-section D5 in the treatment region 121. This distal oval configuration can have dimensions of about 0.025 inches by 0.012 inches, about 0.020 inches by 0.010 inches, or about 0.021 inches by 0.013 inches. The wall thickness can vary along the length, from about 0.004 inches in the interface region 117, to about 0.008 inches in the transition region 119, and about 0.004 to 0.008 inches in the treatment region 121.

The oval cross-sectional geometry can provide increased surface area for engagement with clot material compared to a circular cross-section of similar circumference. For instance, sidewall openings can be formed along the long side of the oval shaped surface. Additionally or alternatively, the oval shape can help maintain a lower profile in one dimension while providing enhanced aspiration capability through the larger dimension of the oval. In some implementations, an oval cross-section along the treatment region 121 can accommodate larger openings than in embodiments having strictly circular cross-sections. The larger openings may enhance clot engagement and allow the oval cross-section to separate more of the clot from the vessel wall as compared to embodiments having circular cross-sections.

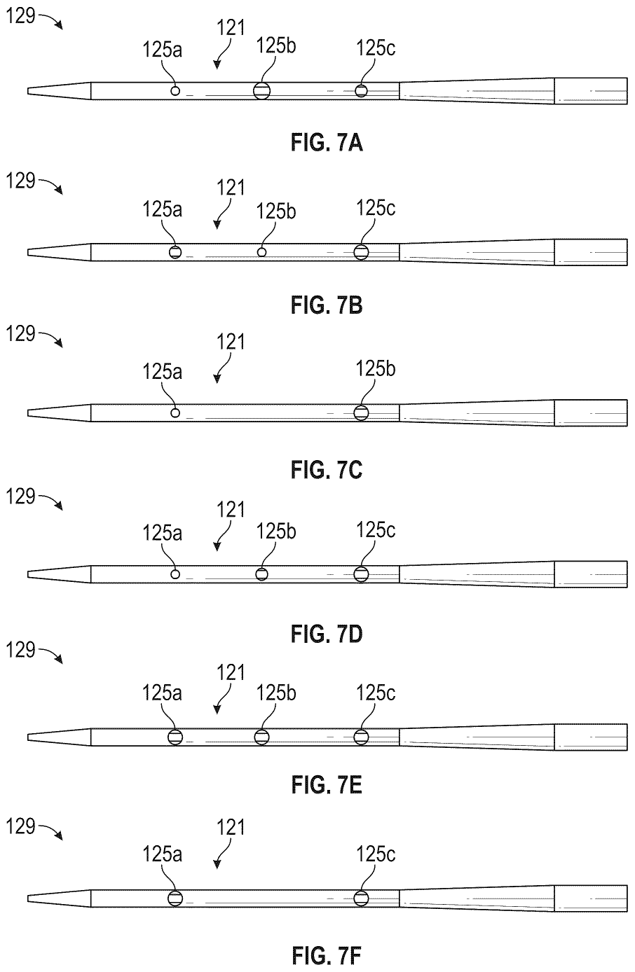

FIGS. 7A-7F illustrate various configurations of a distal shaft 129 having different arrangements of sidewall openings 125 in the treatment region 121. In some implementations, the sidewall openings (designated as 125a, 125b, and/or 125c) can be arranged in different patterns along the treatment region 121 to achieve desired aspiration characteristics.

FIG. 7A shows a first configuration where three sidewall openings 125a, 125b, and 125c are spaced substantially equidistantly along the treatment region 121. According to some implementations, these openings can be aligned along a common radial position of the distal shaft 129. As illustrated, the middle opening 125b is largest, followed by a smaller opening 125c that is proximalmost, and a smallest opening 125a positioned distalmost. FIG. 7B illustrates a similar arrangement to FIG. 7A, but with modified sizes of the openings 125. As illustrated, the proximalmost opening 125c and distalmost opening 125a can have similar sizes, but the middle opening 125b is smaller.

FIG. 7C depicts a configuration with two sidewall openings 125a and 125b that are spaced further apart from one another than those of FIGS. 7A and 7B. In FIG. 7C, the proximalmost opening 125b is also larger than the distalmost opening 125a. According to various implementations, these openings can be sized differently, with one opening having a larger dimension than the other. In one embodiment, proximalmost opening 125b is a circular hole having a diameter of about 0.035 inch, i.e., 0.035±0.005 inch, distalmost opening 125a is a circular hole having a diameter of about 0.020 inch, i.e., 0.020±0.005 inch, and the distance between proximalmost opening 125b and distalmost opening 125a is about 8 mm, i.e., 7-10 mm.

FIG. 7D shows another three-opening configuration where the opening sizes decreases along the distal direction, such that proximalmost opening 125c is largest, followed by middle opening 125b, and finally distalmost opening 125a is smallest. In some implementations, this variation in opening size can help optimize aspiration characteristics along the length of the treatment region 121. In one embodiment, proximalmost opening 125c is a circular hole having a diameter of about 0.035 inch, i.e., 0.035±0.005 inch, middle opening 125b is a circular hole having a diameter of about 0.035 inch, i.e., 0.035±0.005 inch, distalmost opening 125a is a circular hole having a diameter of about 0.020 inch, i.e., 0.020±0.005 inch, and the distance between proximalmost opening 125c and distalmost opening 125a is about 8 mm, i.e., 7-12 mm. FIG. 7E presents a configuration similar to FIG. 7D but with substantially identical size openings across each of the openings 125a, 125b, and 125c.

FIG. 7F shows a configuration with two openings 125a and 125c having substantially identical opening sizes are spaced further apart along the treatment region 121 without an intervening middle opening. The various configurations of sidewall openings 125 can provide different advantages depending on the specific application or treatment scenario. Multiple openings can create multiple points of engagement with clot material, while variations in opening size and spacing can help optimize aspiration force distribution and clot engagement characteristics.

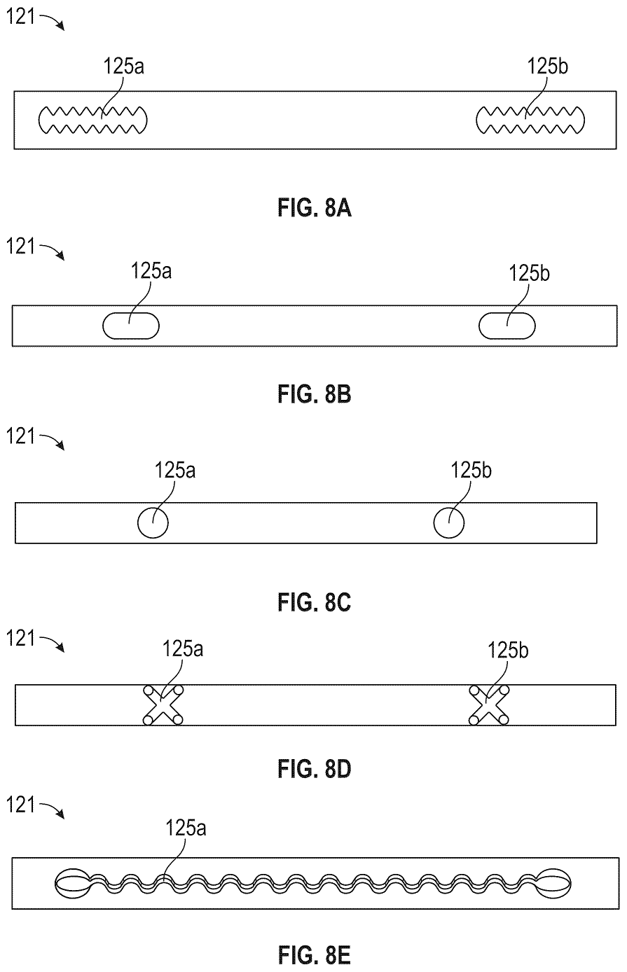

FIGS. 8A-8E illustrate various geometric configurations for the sidewall openings 125 formed in the treatment region 121 of the distal shaft. According to some implementations, these different opening shapes can provide particular benefits for clot engagement and aspiration characteristics.

FIG. 8A shows sidewall openings 125a and 125b configured as wave-shaped patterns. In some implementations, each opening includes multiple wave segments that can create varying contact surfaces with clot material. FIG. 8B depicts sidewall openings 125a and 125b formed as elongated slots. According to some implementations, these slots can be oriented parallel to the longitudinal axis of the distal shaft, providing extended regions for fluid communication between the lumen and the exterior of the treatment device. FIG. 8C illustrates sidewall openings 125a and 125b configured as circular holes. In some implementations, these circular openings can provide uniform aspiration characteristics around their perimeter. FIG. 8D shows sidewall openings 125a and 125b formed in an X-shaped or cross pattern. According to some implementations, these crossed openings can create multiple edges for engagement with clot material while maintaining structural integrity of the treatment region 121. FIG. 8E depicts a configuration where sidewall openings 125a and 125b take the form of an extended sinusoidal or undulating strip pattern. In some implementations, this continuous undulating pattern can provide increased surface area for clot engagement while the sinusoidal geometry helps maintain structural support along the treatment region 121. The various opening geometries can be selected based on specific treatment requirements, such as desired aspiration force profiles, clot engagement characteristics, or structural considerations of the distal shaft 129.

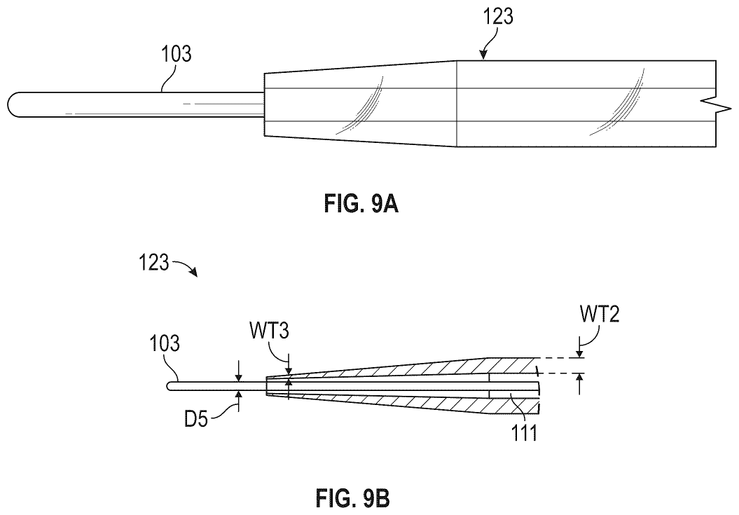

FIGS. 9A and 9B illustrate an example distal tip configuration of a treatment device in accordance with implementations of the present technology. FIG. 9A shows a side view of a distal tip region 123 with a guidewire 103 extending therethrough. FIG. 9B provides a cross-sectional side view of the same region, illustrating internal features of the distal tip region 123.

In various implementations, the distal tip region 123 includes a tapered configuration that facilitates navigation through tortuous and/or small-diameter vessels, such as those found in the cerebral vasculature. The distal tip region 123 can be characterized by a gradual reduction in both outer dimension and lumen dimension. According to some examples, the wall thickness transitions from a first wall thickness WT2 at a proximal portion of the distal tip region 123 to a second, reduced wall thickness WT3 at a more distal portion. In some implementations, WT2 can be between about 0.005 inches, while WT3 can be about 0.004 inches.

The lumen 111 extending through the distal tip region 123 can taper to a reduced dimension D5 at or near the distalmost portion of the distal tip region 123. According to some implementations, D5 can be selected to be just large enough to slidably accommodate the guidewire 103 while maintaining a minimal outer profile. For example, in some implementations, D5 can be about 0.010 inches, which can accommodate a 0.010-inch guidewire, or may also be able to accommodate a 0.012-inch or 0.014-inch guidewire due to the relatively thin and flexible nature of the distal tip walls at WT3. The combination of the tapered configuration and reduced wall thickness can provide enhanced flexibility at the distal tip region 123, which can facilitate atraumatic navigation through vessels while maintaining sufficient column strength for device advancement.

Additionally or alternatively, the tapered configuration of the distal tip region 123 can help reduce the likelihood of the treatment device becoming caught on anatomical features or vessel irregularities during advancement or retraction. The gradual taper can provide a smooth transition with the guidewire that can help the device navigate through curved or branching vessels while maintaining its intended path along the guidewire 103.

### C. Example Joint Configurations Connecting Proximal and Distal Shafts

According to various implementations, the treatment device 101 can be assembled by joining together different shaft components having distinct mechanical properties suited for their respective functions. For instance, the distal shaft 129 can be formed of a relatively flexible material (e.g., silicone or other suitable elastomeric polymer) to facilitate navigation through tortuous anatomy, while the proximal shaft 131 can incorporate structural reinforcement (e.g., a metallic hypotube, metallic braid, or other suitable reinforcing structure, optionally provided along with suitable jackets, coatings, liners, etc.) to provide enhanced mechanical properties such as column strength and kink resistance. The junction between these components presents particular engineering challenges, as it must maintain sufficient bond strength to prevent separation during use while also providing appropriate flexibility for navigation and maintaining consistent lumen dimensions to avoid disrupting flow characteristics. Various implementations of the present technology provide different approaches for creating this junction, each offering particular advantages for specific use cases. As described in more detail below, example joint configurations can include adhesive bonds, mechanical interlocking features, thermal bonding arrangements, and/or combinations thereof.

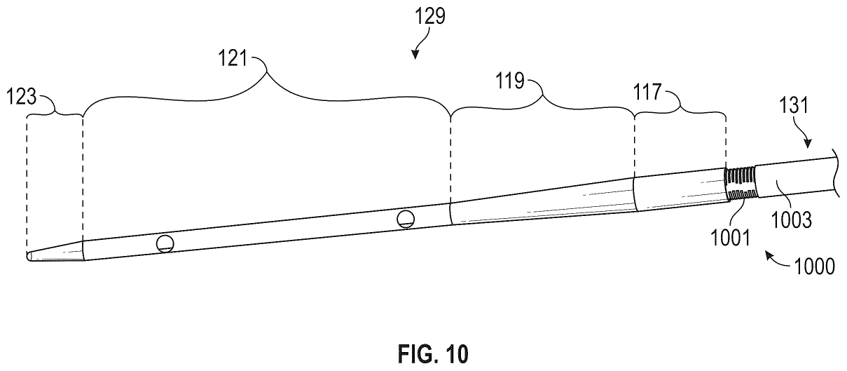

FIG. 10 illustrates an example joint 1000 for connecting the distal shaft 129 and proximal shaft 131 of a treatment device 101 in accordance with implementations of the present technology. The figure shows the components in a partially assembled state to illustrate the relationship between various features of the joint 1000. According to some implementations, the proximal shaft 131 can include a metallic hypotube 1001 having an exposed portion configured to be received within the interface region 117 of the distal shaft 129. The proximal shaft 131 can further include a jacket 1003 (e.g., formed of a polymeric material) disposed over a portion of the hypotube 1001. In various examples, the jacket 1003 can extend proximally from the intended joint location.

During assembly, an adhesive can be applied to the exposed portion of the hypotube 1001. After adhesive application, the hypotube 1001 can be slidably advanced into the interface region 117 of the distal shaft 129. According to some implementations, in the fully assembled configuration, a distal end of the jacket 1003 can be positioned to abut or be partially received within a proximal end of the interface region 117, creating a substantially smooth transition between these components. This arrangement can help prevent the formation of edges or transitions that might impede advancement of the treatment device 101 through tortuous anatomy.

As noted previously, the distal shaft 129 can include various regions progressing distally from the interface region 117, including the transition region 119, treatment region 121 (which can include one or more sidewall openings as described elsewhere herein), and distal tip region 123. The joint 1000 can provide a secure connection between the proximal shaft 131 and the distal shaft 129 while maintaining appropriate flexibility characteristics for navigation through vessels.

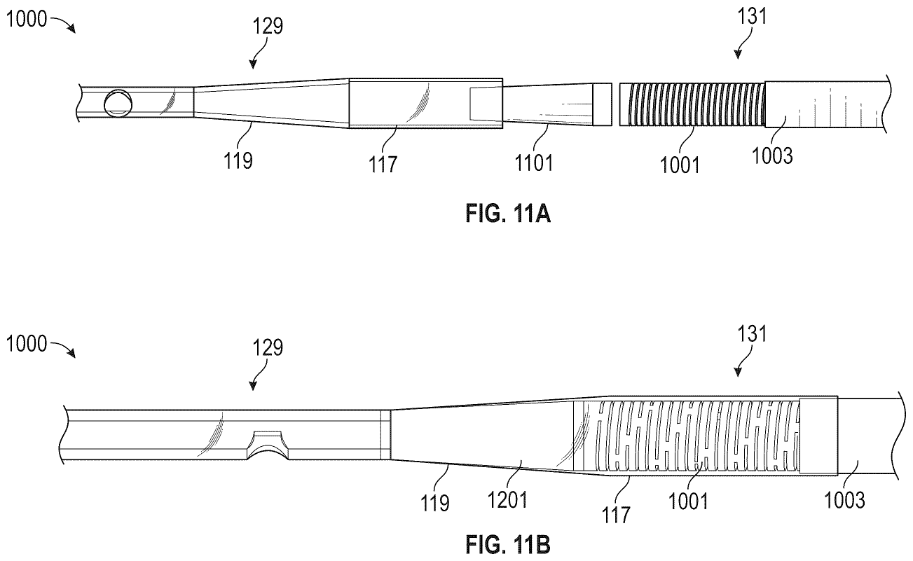

FIGS. 11A and 11B illustrate another example joint 1000 for connecting the distal shaft 129 to the proximal shaft 131 in accordance with implementations of the present technology. FIG. 11A shows an exploded view of the components in an unassembled state, while FIG. 11B shows the components in their assembled configuration.

According to some implementations, this joint 1000 includes a tapered insert 1101 in addition to the features described above with reference to FIG. 10. As shown in FIG. 11A, the proximal shaft 131 can include a metallic hypotube 1001 having an exposed portion, and a jacket 1003 disposed over a portion of the hypotube 1001. The tapered insert 1101 is configured to be received within the transition region 119 of the distal shaft 129, distal to where the exposed portion of the hypotube 1001 will be received within the interface region 117.

During assembly, adhesive can be applied to one or more of: the exposed portion of the hypotube 1001, the tapered insert 1101, or corresponding receiving portions of the distal shaft 129. The components can then be brought together as shown in FIG. 11B, with the tapered insert 1101 and exposed hypotube portion 1001 being slidably advanced into the distal shaft 129. According to some implementations, in the assembled state, the tapered insert 1101 can provide additional structural support within the transition region 119 while also helping to maintain appropriate lumen dimensions through the joint.

As illustrated in FIG. 11B, when assembled, the jacket 1003 can abut or be partially received within the proximal end of the interface region 117, providing a substantially smooth transition between components. The tapered configuration of the insert 1101 can help provide a gradual transition in mechanical properties (e.g., stiffness) between the relatively rigid proximal shaft 131 and the more flexible distal shaft 129. Additionally or alternatively, the tapered insert 1101 can help maintain consistent inner lumen dimensions through the joint region to avoid disrupting flow characteristics during operation of the treatment device.

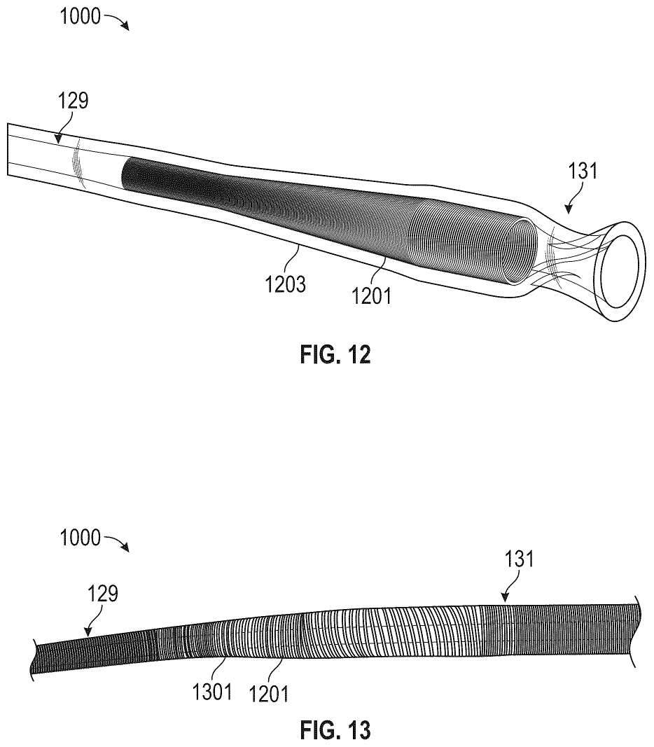

FIG. 12 illustrates another example joint 1000 for connecting the distal shaft 129 to the proximal shaft 131 in accordance with implementations of the present technology. In this configuration, the joint 1000 incorporates a tapered coil 1201 which serves as a reinforcement and is surrounded by an overmolded material 1203. According to some implementations, the coiled reinforcement structure can be coupled to the proximal shaft 131 be configured to extend into distal shaft 129. The tapered coil 1201 can provide a gradual transition in mechanical properties, with the coil diameter decreasing in the distal direction. In various examples, the coiled reinforcement structure can be formed from a metallic material (e.g., stainless steel, Nitinol, or other suitable material) to provide enhanced structural support while maintaining appropriate flexibility characteristics.

The overmolded material 1203 extending circumferentially around the joint 1000 encapsulates the tapered coil 1201 and creates a secure connection between the proximal shaft 131 and distal shaft 129. The overmolded material 1203 can be formed of a polymeric material selected to be compatible with both the proximal and distal shaft materials. According to some implementations, this arrangement can provide a strong and durable connection while maintaining appropriate flexibility for navigation through tortuous anatomy. The gradual taper of the coil 1201 can help prevent abrupt transitions in mechanical properties that might otherwise create areas susceptible to kinking or other failure modes during use.

The coiled configuration can provide several advantages compared to other joint arrangements. For instance, the spacing between adjacent coils can allow some degree of flexion while maintaining column strength and resistance to kinking. Additionally or alternatively, the coiled structure can help maintain consistent lumen dimensions through the joint region while providing a secure attachment mechanism between the proximal shaft 131 and distal shaft 129.

FIG. 13 illustrates another example joint 1000 for connecting the distal shaft 129 to the proximal shaft 131 in accordance with implementations of the present technology. Similar to the configuration shown in FIG. 12, this implementation incorporates a tapered coil 1201 as a reinforcement structure. However, in this configuration, the joint 1000 is secured using a reflowable material 1301 (e.g., thermoplastic polyurethane or other suitable thermoplastic polymer) rather than an overmolded structure.

According to some implementations, the reflowable material 1301 can be disposed along the coiled reinforcement structure and then subjected to controlled heating to cause the material to reflow into and around the coils. Upon cooling, the reflowed material 1301 can create a secure mechanical and chemical bond between the proximal shaft 131 and the distal shaft 129. The reflowed material 1301 can penetrate between adjacent coils of the reinforcement structure, creating a more intimate connection between components compared to alternative joining methods.

In various examples, the tapered coil 1201 can provide a gradual transition in mechanical properties, with the coil diameter decreasing in the distal direction. This tapering configuration, in combination with the reflowed material 1301, can help prevent abrupt transitions in flexibility that might otherwise create points susceptible to kinking during navigation through tortuous anatomy. Additionally or alternatively, the spacing between adjacent coils can allow the reflowable material 1301 to flow between and around the coils during the reflow process, potentially creating a more secure bond compared to alternative joining techniques.

The use of a reflowable material 1301 can provide several advantages compared to other joint arrangements. For instance, the reflow process can create a more uniform and continuous bond along the length of the joint 1000, potentially reducing the likelihood of separation or failure during use. Additionally or alternatively, appropriate selection of the reflowable material 1301 can provide desired combinations of bond strength and flexibility characteristics suited for particular applications.

### D. Example Radiopaque Marker Configurations

According to various implementations, the treatment device 101 can include one or more radiopaque markers positioned at strategic locations to facilitate visualization under fluoroscopic imaging during device navigation and operation. These markers can take various forms, such as bands, coils, tubes, or other suitable geometries, and can be formed from radiopaque materials including platinum, tungsten, barium sulfate, or combinations thereof. The markers can be positioned at locations of particular interest to the physician, such as adjacent to sidewall openings 125, at transitions between different shaft regions, or at the distal tip. In some implementations, the markers can be disposed within the lumen 111, while in other examples the markers can be positioned around the outer surface of the treatment device 101. Additionally or alternatively, certain markers can incorporate specialized geometries that align with functional features of the device, such as a tubular marker having a sidewall opening that aligns with a corresponding sidewall opening of the treatment device 101. When using the treatment device 101 under fluoroscopic guidance, these markers can help the physician understand the precise location and orientation of key features, facilitate accurate positioning relative to a target site such as a thrombus, and help confirm proper device operation such as engagement between sidewall openings 125 and surrounding tissue.

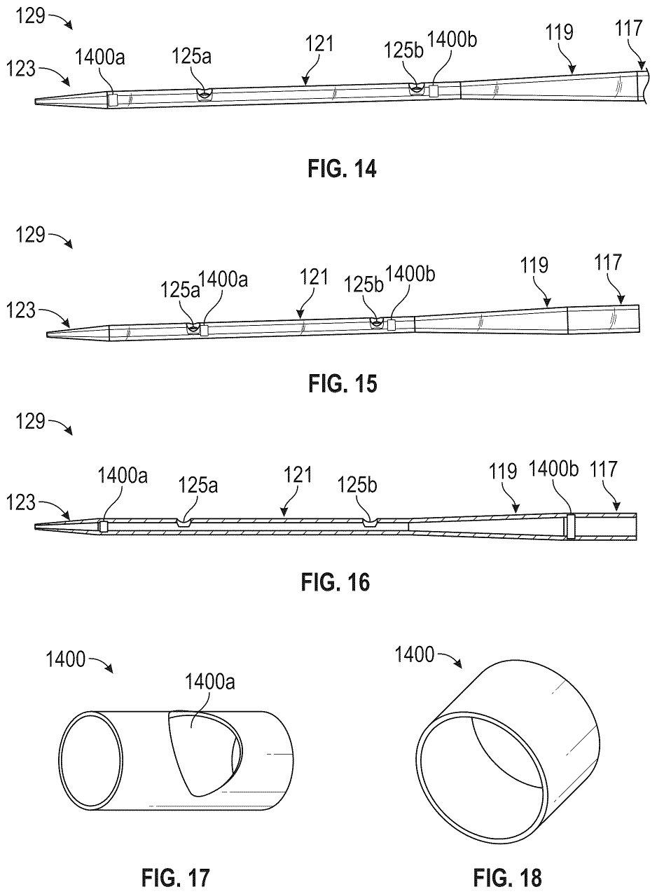

FIGS. 14-16 illustrate various configurations of radiopaque markers 1400 positioned along the distal shaft 129 in accordance with implementations of the present technology. FIGS. 17 and 18 show detailed views of example radiopaque markers 1400 that can be used in these and other marker arrangements.

According to some implementations, FIG. 14 shows a first marker arrangement where radiopaque markers 1400a and 1400b are positioned within the lumen, with first marker 1400a positioned at the end of the treatment region 121 and the second marker 1400b positioned at a position proximal to the proximal sidewall opening 125b. This configuration can help the physician visualize the location of the effective treatment zone (e.g., including both the first opening 125a and second opening 125b) under fluoroscopy, facilitating proper alignment with a treatment site such as a thrombus.

FIG. 15 depicts an alternative marker arrangement where the markers 1400a and 1400b are each positioned directly proximal to the respective sidewall openings 125a and 125b. This allows for visualization of exact locations of the sidewall openings 125a and 125b under fluoroscopy, facilitating alignment with anatomical features at the treatment site.

FIG. 16 shows another example configuration where a first marker 1400a is positioned near the distal tip region 123, while a second marker 1400b is positioned at or near the interface region 117. This arrangement can help the physician understand the overall position and orientation of the distal shaft 129 during navigation and treatment.

FIG. 17 provides a detailed view of an example radiopaque marker 1400 in the form of a tubular section having a side opening 1401 formed therein. In operation, this marker 1400 can be positioned within the lumen 111 of the treatment device 101 and the sidewall opening 1401 of the marker 1400 can be aligned with the sidewall opening 125 of the treatment device 101, thereby providing a radiopaque marker fully aligned with the sidewall opening 125 without interrupting fluid flow therethrough.

FIG. 18 illustrates another example radiopaque marker 1400 in the form of a band, annulus, or short tubular segment. In various examples, this marker can be positioned within the lumen 111, crimped over the outer surface of the treatment device 101, or any other suitable position.

The radiopaque markers 1400 can be formed from various radiopaque materials, such as platinum, tungsten, barium sulfate, or combinations thereof. Additionally or alternatively, the markers can be secured to the treatment device 101 using various attachment mechanisms, such as adhesive bonding, mechanical crimping, or embedding within the sidewall material. In some implementations, the specific marker configuration and attachment mechanism can be selected based on the desired combination of visualization, flexibility, and structural characteristics for particular applications. Although the illustrated examples has two markers 1400, in various implementations there may be 1, 3, 4, 5, 6, 7, 8 or more radiopaque markers.

### II. Example Treatment Methods

The systems and devices described herein can be used in a variety of medical procedures and treatment approaches. While many implementations involve thrombectomy procedures in which the treatment device 101 is used to aspirate and remove thrombus or other occlusive material from blood vessels, the technology can be applied to various other treatment scenarios. According to some implementations, the treatment device 101 can be used in conjunction with flow monitoring equipment to provide real-time feedback about device operation, such as confirming engagement between the sidewall openings and surrounding tissue or identifying potential issues like partial or complete disengagement during clot retrieval. Additionally or alternatively, rather than applying suction through the lumen 111, positive pressure can be applied to deliver fluids (e.g., saline, medicaments, contrast media, etc.) to a treatment site via the sidewall openings 125. In some examples, the treatment device 101 can be used to visualize clot length and morphology under fluoroscopy to help inform device selection or treatment strategy. The following sections describe various methods of using the treatment device 101, though it should be understood that other treatment approaches incorporating features of the present technology are possible.

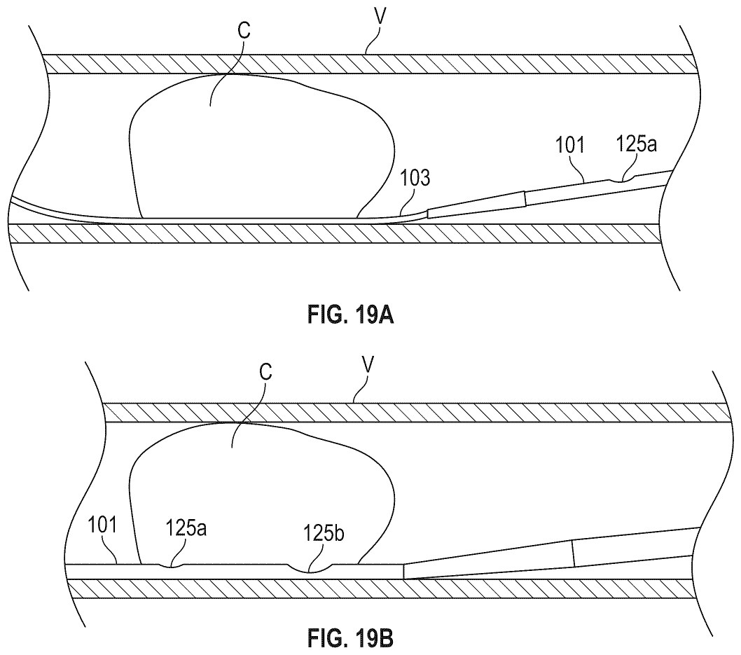

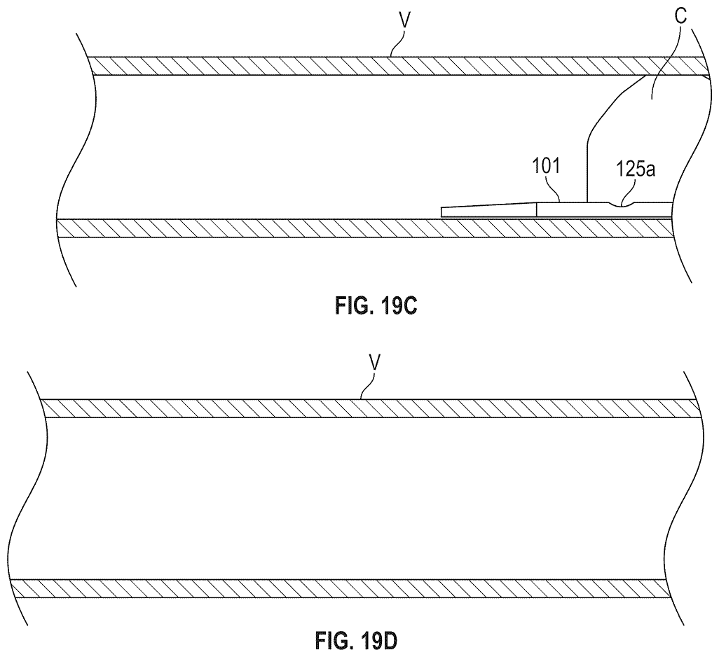

FIGS. 19A-19D illustrate an example method of using the treatment device 101 to remove a clot C from a vessel V in accordance with implementations of the present technology. As shown in FIG. 19A, a guidewire 103 can first be navigated through the vessel V such that it crosses through the clot C, traversing the clot between the vessel wall and the clot. According to some implementations, the treatment device 101 can then be slidably advanced over the guidewire 103 in a distal direction toward the treatment site. In various examples, the tapered configuration of the distal tip region and the relatively small profile of the treatment device 101 can facilitate advancement through potentially tortuous anatomy to reach the clot C.

FIG. 19B illustrates the treatment device 101 having been advanced over the guidewire such that it crosses the clot C, traveling the clot between the vessel wall and the clot, with sidewall openings 125a and 125b positioned to overlap with portions of the clot C. In some implementations, radiopaque markers (not shown) can help confirm proper positioning of the sidewall openings 125a and 125b relative to the clot C under fluoroscopic visualization. Once properly positioned, the guidewire may be left in place or may be removed, and negative pressure can be supplied to the lumen of the treatment device 101, causing portions of the clot C to be drawn into engagement with the sidewall openings 125a and 125b.

According to various implementations, FIG. 19C shows the treatment device 101 being retracted proximally with the clot C secured in engagement via the applied negative pressure. Optionally, the treatment device 101 and engaged clot C can be withdrawn into a surrounding catheter (not shown) positioned proximal to the treatment site. The multiple points of engagement between the clot C and the sidewall openings 125a and 125b can help maintain secure attachment during withdrawal while reducing the likelihood of fragmentation or disengagement. FIG. 19D shows the vessel V after successful removal of the clot C using the treatment device 101, illustrating restoration of the vessel lumen.

As noted previously, the treatment device 101 can be used in conjunction with a supporting catheter (e.g., a guide catheter, access catheter, or other suitable catheter) to enhance the efficacy of clot removal procedures. For instance, after engaging a clot via the sidewall openings 125, the treatment device 101 can be withdrawn into the supporting catheter while maintaining aspiration through the lumen 111. In some implementations, this configuration allows the treatment device 101 to continue functioning as an aspiration catheter even while positioned within the lumen of the supporting catheter. In this configuration, as the treatment device 101 is withdrawn into the lumen of the supporting catheter, and the clot blocks the opening of the support catheter, proximal opening 125b may be pulled away from the clot and proximally into the support catheter. In such a case, the aspiration through opening 125b creates aspiration proximally to the clot and across the lumen of the support catheter, causing the clot to be ingested into the support catheter or at least firmly corked into the support catheter. Additionally or alternatively, the supporting catheter can be coupled to a separate aspiration source to provide supplemental aspiration through the annular space between the treatment device 101 and the supporting catheter. This dual-aspiration arrangement can help capture any larger clot fragments or smaller pieces that might otherwise separate from the primary clot during withdrawal. According to some examples, if clot material does become disengaged from the treatment device 101 during withdrawal, the sustained aspiration through both the treatment device 101 and supporting catheter can help prevent such fragments from embolizing distally. In various implementations, this combined use of the treatment device 101 and supporting catheter can provide a more comprehensive approach to clot removal compared to conventional single-catheter techniques.

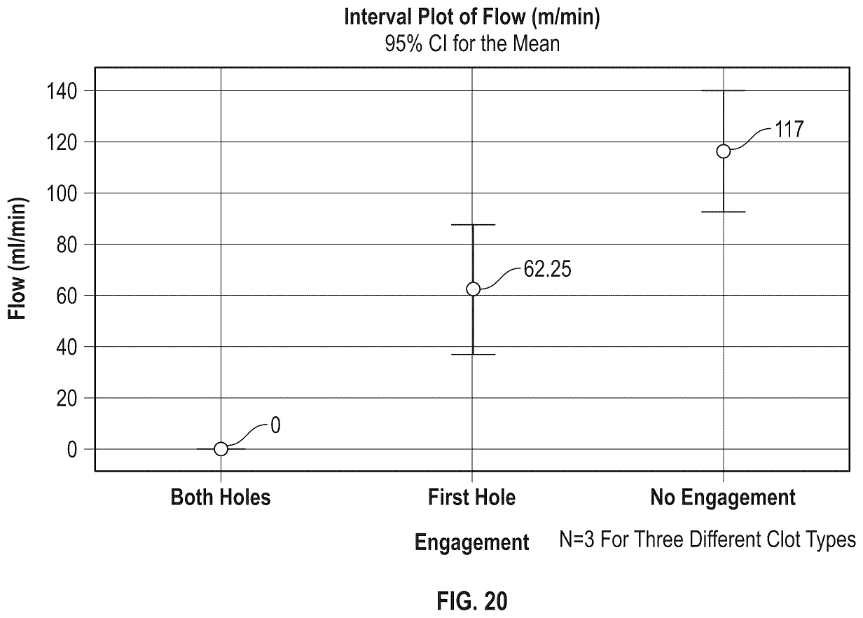

According to various implementations, during operation of the treatment device 101, a flow monitor 109 (FIG. 1) coupled to the treatment device 101 can provide real-time feedback about engagement between the treatment device 101 and surrounding clot material. As illustrated in FIG. 20, the measured flow rate through the lumen can vary significantly depending on the degree of engagement between the clot and the sidewall openings. When there is no guidewire and no engagement between the clot and the treatment device 101, relatively high flow rates of approximately 117 ml/min can be observed as fluid flows freely through both sidewall openings. This may trigger an alert to the user that an undesirable amount of blood is being removed from the patient, or may act as part of a control system to shut off aspiration after a predetermined amount of time. In contrast, when there is no guidewire and the clot engages with a single sidewall opening (“First hole”), the measured flow rate drops to approximately 62.25 ml/min due to partial occlusion of the aspiration path. Upon full engagement, where the clot material engages with both sidewall openings (“Both holes”), the measured flow rate approaches zero as the clot substantially blocks fluid flow through the openings. Because of the small size of dimension D5 at the distal end of the distal tip region 123, this is true even with no guidewire present. According to some implementations, this flow rate data can help the physician confirm proper engagement before attempting to withdraw the treatment device 101, potentially improving procedural success rates and reducing the likelihood of clot disengagement during retrieval. Additionally or alternatively, sudden changes in measured flow rate during withdrawal can alert the physician to potential disengagement or fragmentation of the clot, allowing for immediate procedural adjustments if needed.

According to various implementations, the treatment device 101 can be used to deliver fluid to a treatment site through the sidewall openings 125 instead of, or in addition to, performing aspiration. For example, a fluid source 107 (FIG. 1) can be coupled to the proximal end of the treatment device 101 to supply positive pressure through the lumen 111, causing fluid to exit through the sidewall openings 125 at the treatment site. The fluid may include saline, contrast media, therapeutic agents, thrombolytics, anti-inflammatory medications, or other suitable medicaments. In some implementations, the multiple sidewall openings 125 can provide distributed delivery of the fluid around the circumference of the treatment site compared to single-opening devices. Additionally or alternatively, when the treatment device 101 is advanced through or adjacent to a clot, delivery of contrast media through the sidewall openings 125 can help visualize the extent and morphology of the clot under fluoroscopy, potentially informing decisions about device selection or treatment strategy. The relatively small profile of the distal shaft 129 and the tapered configuration can allow the treatment device 101 to access relatively distal treatment sites for targeted fluid delivery, while the larger lumen dimensions in the proximal shaft 131 can help minimize pressure drop and maintain desired flow rates through the device.

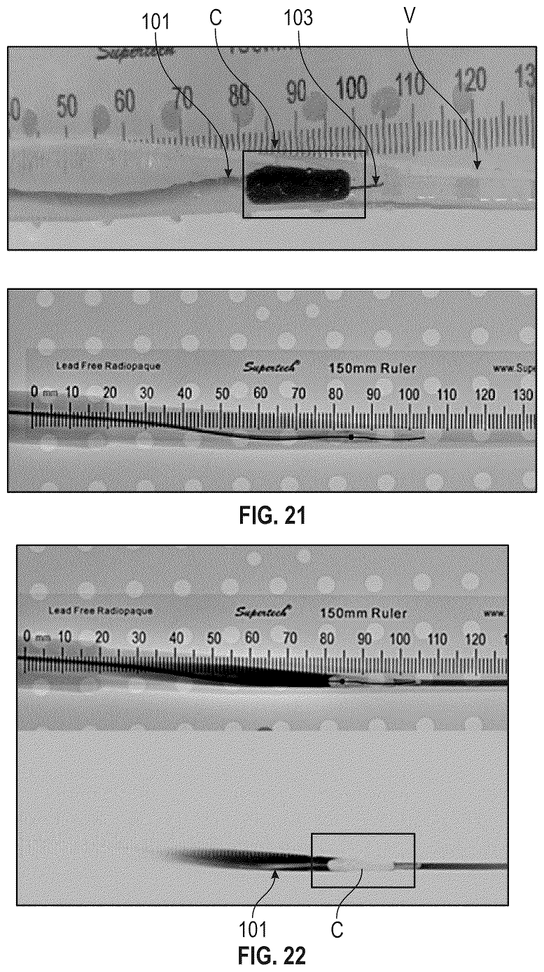

FIGS. 21 and 22 illustrate techniques for visualizing and characterizing clot morphology using the treatment device in accordance with implementations of the present technology. FIG. 21 includes two views of a test configuration in which a clot C is positioned within an artificial vessel V. In the upper image (a photographic view), the treatment device 101 is shown extending across the clot C, with a guidewire 103 extending through and beyond both the treatment device 101 and the clot C. In the lower image (a fluoroscopic view), the clot C is substantially radiolucent and thus not readily visible under standard fluoroscopic imaging.

In some implementations, the treatment device 101 may be used to visualize or measure an obstruction, such as the clot C depicted in FIGS. 21-22. A physician may advance the treatment device 101, optionally over a guidewire 103, to a position where the treatment region 121 spans the clot C. The proximal end of the clot C may be identified through previous contrast injections or, additionally or alternatively, by using the treatment device 101 itself. To visualize both the proximal and distal ends, a fluid source 107 may be coupled to the proximal end 101a of the treatment device 101 to deliver contrast media. In this arrangement, the treatment device 101 may be positioned such that a proximal sidewall opening, such as 125b, is located proximal to the clot C, and a distal sidewall opening, such as 125a, is located distal to the clot C. When positive pressure is applied, the delivered contrast media exits the proximal sidewall opening 125b to define the proximal end of the clot C, and simultaneously exits the distal sidewall opening 125a to define the distal end of the clot C. This method, depicted generally in FIG. 22, allows the clot C to appear as a relative void or lighter region between the proximal and distal contrast-filled regions, which may facilitate a measurement of the clot length. This procedure may be performed with the guidewire 103 remaining in place, or after the guidewire 103 has been removed.

Additionally or alternatively, a similar method for visualizing the clot C may be performed after the guidewire 103 has been proximally retracted and removed from the lumen 111 of the treatment device 101. In this configuration, the treatment device 101 may be positioned such that at least one proximal sidewall opening 125b is located proximal to the clot C, while the distal opening 127 at the distal tip region 123 is positioned distal to the clot C. When contrast media is delivered from the fluid source 107, the fluid exits the proximal sidewall opening 125b to visualize the proximal face of the clot C. The contrast media also travels the length of the lumen 111 and exits through the distal opening 127 to visualize the area distal to the clot C. This again allows for the identification of the clot's length and morphology based on the resulting radiolucent void between the two zones of contrast.

FIG. 22 depicts fluoroscopic views of the test configuration after delivery of a contrast medium through the lumen of the treatment device 101. As shown in FIG. 22, because the treatment device 101 extends across the clot C, contrast medium can be delivered both proximal and distal to the clot C. The contrast medium appears as darkened regions in the fluoroscopic views, with the clot C appearing as a relative void or lighter region between the proximal and distal contrast-filled regions. According to some implementations, this configuration allows for measurement of clot length under fluoroscopy by measuring the length of the contrast-void region between the proximal and distal contrast-filled regions. In various examples, physicians can use this length information to inform device selection or treatment strategy. Additionally or alternatively, the contrast delivery can provide information about clot morphology, density, or other characteristics that may be useful for treatment planning.

The ability to deliver contrast medium through and/or around the clot C via the treatment device 101 can provide several advantages compared to conventional approaches. For instance, in some implementations, accurate measurement of clot length can help ensure selection of appropriately sized treatment devices. Additionally or alternatively, the ability to deliver contrast medium both proximal and distal to the clot can help confirm proper positioning of the treatment device 101 relative to the target clot C.

### EXAMPLES

The subject technology is illustrated, for example, according to various aspects described below, including with reference to FIGS. 1-22. Various examples of aspects of the subject technology are described as numbered examples (1, 2, 3, etc.) for convenience. These are provided as examples and do not limit the subject technology.

Example 1. A treatment device comprising: a tubular member having a proximal end region configured to be disposed extracorporeally and a distal end region configured to be disposed at a treatment site, the tubular member comprising: a sidewall defining a lumen extending from the proximal end region to the distal end region; and a plurality of sidewall openings in the distal end region and in fluid communication with the lumen.

Example 2. The treatment device of Example 1, wherein the tubular member comprises an aspiration catheter.

Example 3. The treatment device of any one of Examples 1-2, wherein the tubular member comprises a fluid-delivery catheter.

Example 4. The treatment device of any one of the preceding Examples, wherein at least some of the sidewall openings are spaced apart from one another along an axial direction.

Example 5. The treatment device of any one of the preceding Examples, wherein at least some of the sidewall openings are spaced apart from one another along a radial direction.

Example 6. The treatment device of any one of the preceding Examples, wherein an inner dimension defining the lumen tapers along the length of the lumen.

Example 7. The treatment device of any one of the preceding Examples, wherein the device comprises a proximal region having a first lumen outer dimension and a distal region having a second lumen outer dimension, the second outer dimension smaller than the first outer dimension.

Example 8. The treatment device of Example 7, wherein the treatment device comprises an intermediate region between the proximal region and the distal region, and wherein in the intermediate region the lumen outer dimension tapers from the first lumen outer dimension to the second lumen outer dimension.

Example 9. The treatment device of any one of Examples 7-8, wherein the sidewall openings are disposed along the distal region of the treatment device.

Example 10. The treatment device of any one of Examples 7-9 wherein the treatment device further comprises a distal tip disposed distal to the distal region, the distal tip having a lumen outer dimension that tapers from the second lumen outer dimension to a third lumen outer dimension, wherein the third lumen outer dimension is smaller than the second lumen outer dimension.

Example 11. The treatment device of Example 10, wherein the third lumen outer dimension is configured to slidably receive a guidewire therethrough.

Example 12. The treatment device of any one of Examples 1-11, wherein the treatment device comprises a proximal shaft including the proximal end region configured to be disposed extracorporeally, and a distal shaft coupled to the proximal shaft and including the distal end region configured to be disposed at an intravascular treatment site, wherein the lumen extends through both the proximal shaft and the distal shaft.

Example 13. The treatment device of Example 12, wherein the distal shaft is more flexible than the proximal shaft.

Example 14. The treatment device of any one of Examples 12-13, wherein the proximal shaft comprises a metallic-braid-reinforced catheter.

Example 15. The treatment device of any one of Examples 12-14, wherein the proximal shaft comprises a metallic hypotube.

Example 16. The treatment device of any one of Examples 12-15, wherein the proximal shaft has a lumen dimension of about 0.071″.

Example 17. The treatment device of any one of Examples 12-15, wherein the proximal shaft has a lumen dimension of between about 0.044″ and about 0.088″.

Example 18. The treatment device of any one of Examples 12-17, wherein the distal shaft comprises silicone.

Example 19. The treatment device of any one of Examples 12-18, wherein the distal shaft has a lumen dimension of between about 0.010″ to about 0.055″.

Example 20. The treatment device of any one of Examples 12-19, wherein a wall thickness of the distal shaft tapers in the distal direction.

Example 21. The treatment device of any one of Examples 12-20, wherein the lumen has a substantially constant outer dimension in the proximal shaft and a distally tapering outer dimension in the distal shaft.

Example 22. The treatment device of any one of Examples 12-21, wherein the distal shaft lumen has an oval cross-sectional shape along at least a portion of its length.

Example 23. The treatment device of any one of Examples 12-22, wherein the distal shaft has an oval shaped outer dimension, and wherein at least one of the sidewall openings is elongated along a direction parallel to the major axis of the oval.

Example 24. The treatment device of any one of Examples 12-23, wherein a proximal end of the distal shaft is connected to a distal end of the proximal shaft at a joint.

Example 25. The treatment device of Example 24 wherein the distal end of the proximal shaft is least partially received within the proximal end of the distal shaft.

Example 26. The treatment device of Example 24 or 25, wherein the joint comprises an adhesive.

Example 27. The treatment device of any one of Examples 24-26, wherein the joint comprises a tubular tapered insert, the tapered insert comprising a proximal end abutting the distal end of the proximal shaft, wherein the proximal end of the distal shaft extends over the tapered insert and the distal end of the proximal shaft.

Example 28. The treatment device of any one of Examples 24-26, wherein the joint comprises a tapered coil extending from a distal end of the proximal shaft, wherein the proximal end of the distal shaft extends over the tapered coil and the distal end of the proximal shaft.

Example 29. The treatment device of Example 28, wherein the joint further comprises an overmolded material extending circumferentially around the joint.

Example 30. The treatment device of Example 28, wherein the joint further comprises a reflowable material (e.g., thermoplastic polyurethane) coupled to the coil.

Example 31. The treatment device of any one of Examples 1-30, wherein a first sidewall opening has a first opening size and a second sidewall opening has a second opening size larger than the first.

Example 32. The treatment device of any one of Examples 1-31, wherein each of the sidewall openings are axially aligned and disposed on the same radial side of the tubular member.

Example 33. The treatment device of any one of Examples 1-32, wherein at least one of the sidewall openings has an opening shape that is one or more of: circular, elliptical, oval, axially elongated, circumferentially elongated, X-shaped, star-shaped, sinusoidal, undulating, jagged, curved, or rectilinear.

Example 34. The treatment device of any one of Examples 1-33, further comprising one more radiopaque markers disposed along the tubular member.

Example 35. The treatment device of Example 34, further comprising a plurality of radiopaque markers disposed along the tubular member, wherein at least some of the radiopaque markers are axially spaced apart from one another.

Example 36. The treatment device of Example 34, further comprising a plurality of radiopaque markers disposed along the tubular member, wherein at least some of the radiopaque markers are radially spaced apart from one another.

Example 37. The treatment device of any one of Examples 34-36, wherein at least one radiopaque marker is disposed within the lumen.

Example 38. The treatment device of any one of Examples 34-37, wherein at least one radiopaque marker is disposed over an outer surface of the tubular member.

Example 39. The treatment device of any one of Examples 34-38, wherein at least one radiopaque marker comprises one or more of: platinum, tungsten, or barium sulfate.

Example 40. The treatment device of any one of Examples 34-39, wherein at least one radiopaque marker is positioned adjacent a sidewall opening to facilitate alignment of the sidewall opening with a target location during navigation under fluoroscopy.

Example 41. The treatment device of any one of Examples 34-40, wherein at least one radiopaque marker comprises an annular band or ring.

Example 42. The treatment device of any one of Examples 34-40, wherein at least one radiopaque marker comprises a tube having a sidewall opening formed therein, and wherein the radiopaque marker is disposed such that the radiopaque marker sidewall opening aligns with a sidewall opening of the tubular member.

Example 43. A treatment system comprising: the treatment device of any one of Example 1-42; and a guidewire configured to be slidably disposed within the lumen.

Example 44. A treatment system comprising: the treatment device of any one of Example 1-42; and a suction source configured to supply negative pressure to the lumen to aspirate the treatment site via the sidewall openings.

Example 45. A treatment system comprising: the treatment device of any one of Example 1-42; and a flow monitor configured to monitor a flow rate through the lumen.

Example 46. The treatment system of Example 45, wherein measured flow rate indicates a degree of engagement between the treatment device and a clot.

Example 47. The treatment system of Example 45, wherein the measured flow rate indicates a number of sidewall openings engaged with a clot.

Example 48. The treatment system of Example 45, wherein the system is configured to: obtain a flow rate reading via the flow monitor; and based at least in part on the flow rate reading, evaluate engagement between a clot and the treatment device.

Example 49. The treatment system of Example 48, wherein evaluating engagement between the clot and the treatment device comprises identifying conditions of: no engagement, partial engagement, or full engagement.

Example 50. A treatment system comprising: the treatment device of any one of Example 1-42; and a fluid source configured to supply fluid to the lumen to be delivered to the treatment site via the sidewall openings or through the distal tip.

Example 51. A method comprising: disposing the treatment device of any one of Example 1-42 at or adjacent a treatment site; and supplying negative pressure to the lumen to aspirate the treatment site.

Example 52. A method comprising: disposing the treatment device of any one of Example 1-42 at or adjacent a treatment site; and delivering fluid into the lumen of the treatment device such that fluid exits the sidewall openings or through the distal tip at or adjacent the treatment site.

Example 53. A method comprising: disposing the treatment device of any one of Example 1-42 at an intravascular treatment site such that at least one of the sidewall openings is adjacent a thrombus; and supplying negative pressure to the lumen engage the thrombus via the at least one sidewall opening.

Example 54. The method of Example 53, further comprising moving the treatment device and the engaged thrombus to dislodge the thrombus.

Example 55. The method of Example 53 or 54, further comprising removing the treatment device and the engaged thrombus from the treatment site.

### CONCLUSION

Although many of the embodiments are described above with respect to systems, devices, and methods for treating vessel occlusions in the brain, the technology is applicable to other applications and/or other approaches, such as vessel occlusions elsewhere in the body. As noted herein, in some implementations the treatment devices and systems disclosed herein can be used for delivery of fluid instead of or in addition to aspiration. This can include, for instance, delivery of fluid containing medicament (e.g., any substance used for medical treatment, diagnosis, disease prevention, and/or health promotion). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-22.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system for removing an obstruction from a body lumen, the system comprising:
a treatment device comprising an elongate body defining an aspiration lumen, a distal end region, and a plurality of sidewall openings located in the distal end region and in fluid communication with the aspiration lumen;
a suction source fluidly coupled to the aspiration lumen; and
a flow monitor operatively coupled to the system and configured to measure a flow rate associated with aspiration through the aspiration lumen.

2. The system of claim 1, further comprising a processor communicatively coupled to the flow monitor, the processor configured to evaluate engagement between the obstruction and the plurality of sidewall openings based at least in part on the measured flow rate.

3. The system of claim 2, wherein the processor is configured to identify a status of no engagement, partial engagement, or full engagement.

4. The system of claim 2, further comprising a display communicatively coupled to the processor, the display configured to present a visual indication based on the evaluated engagement.

5. The system of claim 1, wherein the treatment device further comprises a guidewire lumen extending at least partially through the elongate body.

6. The system of claim 1, wherein the treatment device comprises a proximal shaft portion and a distal shaft portion, the distal shaft portion being more flexible than the proximal shaft portion.

7. The system of claim 1, wherein the obstruction is a thrombus within a neurovascular vessel.

8. A method for removing an obstruction from a body lumen, the method comprising:
advancing a treatment device adjacent to the obstruction, the treatment device comprising an elongate body defining an aspiration lumen and a plurality of sidewall openings;
applying suction to the aspiration lumen via a suction source to draw the obstruction toward the plurality of sidewall openings;
monitoring a flow rate within the aspiration lumen using a flow monitor; and
evaluating a status of engagement between the obstruction and the plurality of sidewall openings based on the monitored flow rate.

9. The method of claim 8, wherein evaluating the status of engagement comprises identifying a change in the monitored flow rate corresponding to at least partial occlusion of the plurality of sidewall openings by the obstruction.

10. The method of claim 9, wherein the change in the monitored flow rate is a reduction in the flow rate below a predetermined threshold.

11. The method of claim 8, further comprising generating an audible or visual alert based on the evaluated status of engagement.

12. The method of claim 8, further comprising manipulating the treatment device relative to the obstruction in response to the evaluated status of engagement.

13. The method of claim 12, wherein manipulating the treatment device comprises retracting the treatment device proximally to remove the obstruction from the body lumen.

14. The method of claim 8, wherein advancing the treatment device comprises advancing the treatment device over a guidewire.

15. A treatment device for removing an obstruction from a body lumen, the device comprising:
a proximal shaft comprising a proximal end region and a distal end portion, the proximal shaft comprising a metallic hypotube or a metallic-braid-reinforced catheter;
a distal shaft comprising a proximal end portion and a distal end region, the distal shaft being more flexible than the proximal shaft;
a joint coupling the distal end portion of the proximal shaft to the proximal end portion of the distal shaft;
an aspiration lumen extending from the proximal end region of the proximal shaft to the distal end region of the distal shaft; and
a plurality of sidewall openings in the distal end region of the distal shaft, the plurality of sidewall openings being in fluid communication with the aspiration lumen.

16. The treatment device of claim 15, wherein, at the joint, the distal end portion of the proximal shaft is at least partially received within the proximal end portion of the distal shaft.

17. The treatment device of claim 15, wherein the joint comprises a tubular tapered insert.

18. The treatment device of claim 15, wherein the joint comprises a tapered coil.

19. The treatment device of claim 15, further comprising a guidewire lumen extending at least partially through the proximal shaft and the distal shaft.

20. The treatment device of claim 15, further comprising at least one radiopaque marker positioned on the distal shaft.
